(19) United States
(12) Patent Application Publication
(10) Pub. No.: US 2005/0256394 A1
(43) Pub. Date: Nov. 17, 2005

(54) Electric design device

(75) Inventor: Joerg Hahn, Leinfelden-Echterdingen (DE)

Correspondence Address: DAVIDSON, DAVIDSON & KAPPEL, LLC, 485 SEVENTH AVENUE, 14TH FLOOR, NEW YORK, NY, 10018, US

(73) Assignee: DaimlerChrysler AG, Stuttgart (DE)

(21) Appl. No.: 11/122,698

(22) Filed: May 5, 2005

(30) Foreign Application Priority Data
May 6, 2004 (DE) DE 102004022318.1

(51) Int. Cl. A61B 5/05

(52) U.S. Cl. 600/414

(57) ABSTRACT

The present invention relates to an electronic design device having an output device for displaying a graphic representation, a first and a second input device, and a data processing device. When information is entered via the first input device, a position marker moves on the output device. The device generates and displays on the output device a curve having a fixed section having a fixed path and a freely movable section between the fixed section and the position marker. The device generates the freely movable section so that the freely movable section and the fixed section together form a smooth curve. When information is entered via the second input device, the position of a curve marker located on the curve is shifted along the curve. If, when a fix command is received, the curve marker is located in the freely movable section, the device converts the partial section of the freely movable section between the curve marker and the existing fixed section into a further fixed section.

Priority is claimed to German Patent Application No. DE 10 2004 022 318.1, filed on May 6, 2004, the entire disclosure of which is incorporated by reference herein.

The present invention relates to an electronic device used in design, having a first and a second input device, an output device for displaying a graphic representation, and a data processing device.

### BACKGROUND

Traditionally, designers design lines on an object to be designed, e.g., a motor vehicle, by applying adhesive tape to a drawing or physical model. This adhesive tape indicates the path of the line on the surface of the object.

An electronic device used in design is described in U.S. Pat. No. 6,642,927 B1, the entire disclosure of which is incorporated by reference herein. This device may be used by the designer to design lines without the need for real adhesive tape. Two input devices, which move two position markers on the output device, which is in the form of a computer screen, are used as input devices. The curve created includes a fixed section and a freely movable section. The freely movable section of the curve, which connects the two position markers, is always straight. Curves having curvature may be created by moving both input devices simultaneously. If the first input device is moved while the second input device is held in a stationary position, and if the second input device is then subjected to further movement, curvature is created in the curve. The fixed section may be continuously extended by adding partial sections of the freely movable section.

A device and a method for creating a curve using a data processing device are known heretofore from U.S. Pat. No. 5,588,100, the entire disclosure of which is incorporated by reference herein. Either a free-form section or a section in the form of a polygon is created, as a function of input from an input device such as a mouse. The sections form a continuous curve. The path of the sections depends on what is entered by moving the input device.

### SUMMARY OF THE INVENTION

An object of the present invention is to provide an electronic design device a method for generating and displaying a curve that is processable by a computer with which a more easily processable curve may be created with less input information.

The present invention provides an electronic design device that includes
a first and a second input device,
a data processing device, and
an output device for displaying a graphic representation.

The device according to the present invention is designed so that
information entered via the first input device moves a position marker on the output device,
information entered via the second input device shifts the position of a marker on the curve along the curve, and
the data processing device generates and displays on the output device a curve having a fixed section and a freely movable section as a function of information entered via the two input devices.

The path of the fixed section is fixed and therefore not modifiable. When the position marker is moved, the device generates a freely movable section which connects an endpoint of the fixed section with the position marker. According to the present invention, the device is designed so that the data processing device generates the freely movable section so that the fixed section and the freely movable section together form a smooth curve. In other words, the curve formed by joining together the fixed section and the freely movable section forms a smooth curve. A smooth curve may be expressed using parameters as t→r (t) with t ε [a, b], where r (t) is continuously differentiable to [a, b] and r′ (t)≠0 for all t ε [a, b].

After receiving a fix command, the device extends the fixed section if, when the fix command is received, the marker is located in the freely movable section. The data processing device performs the extension by converting the partial section of the freely movable section between the marker and the existing fixed section into an additional fixed section and joining the existing fixed section and the additional fixed section together to form a single extended fixed section.

The marker is shifted along the curve without this modifying the curve's path. This makes it easier to shift the marker, and subsequent adjustment of the curve is not required. The marker may be shifted right to the beginning or end of the freely movable section or the fixed section, and a user is able to assess the curve without being distracted by a marker and modify it if necessary.

The design device according to the present invention may be implemented using a conventional computer, and special equipment for example for the input devices is not necessary. In particular, the second input device does not need to be designed so that it is able to move the marker or a second position marker across the entire output device. This means the second input device may be more straightforward in design.

Because the curve that is generated is smooth, it approximates more closely the physical reality of, for example, adhesive tape. Moreover, it is easier to automatically subject the curve to further processing. In addition, the freely movable section may be generated automatically or by inputting just a little information via one of the input devices. The device according to the present invention allows a user to enter information either by using the two input devices simultaneously and continuously, or alternatively by using them one after the other. Both methods of input result in a curve that is always smooth.

The input devices are easier to control during use than related-art devices.

### BRIEF DESCRIPTION OF THE DRAWINGS

Below, an exemplary embodiment of the present invention is described in greater detail with reference to the drawings, in which:

FIG. 1 shows an example of system architecture of the design device;

FIG. 2 shows the representation of a curve to be generated, at the start of generation;

FIG. 3 shows the representation of a further curve to be generated, at the start of generation;

FIG. 4 shows the representation of a curve having a fixed section and a freely movable section;

FIG. 5 shows the release of fixing; and

FIG. 6 shows a spatial representation of the curve in two display windows.

### DETAILED DESCRIPTION

The exemplary embodiment relates to an exemplary application of the device and method for designing motor vehicles. Designers design lines on motor vehicles for example for the following:
trim strips,
door joints, door cut-outs,
other joints,
shapes of front or tail lights.

The device includes
an output device for generating a graphic representation,
a first and a second input device, and
a data processing device.

The output device for generating a graphic representation generates the representation in real time. The output device is preferably one of the following:
a computer screen, e.g., a CRT or LCD screen,
an electronic projection surface,
a digital projection device, or
a fast printer.

A plurality of these devices may also be provided as the output devices, e.g., a screen and a digital projection device.

Preferably the first input device is a data processing mouse, e.g., having three buttons, which is connected to the data processing device via a cable or via a cordless link using infrared or radio. In an alternative embodiment, the first input device includes a graphics tablet having a cordless mouse and a data processing pen having a button. Position is detected by a magnetic sensor in the mouse. In this embodiment of the first input device also, data is transferred via cable or radio or infrared from the graphics tablet to the data processing device. A position marker is moved on the output device as a function of movements of and input via the first input device.

Preferably the second input device includes a rotatable button. The device is designed so that when this button is turned the marker is shifted along the curve.

According to a further refinement, the second input device includes a valuator having at least three rotatable buttons. When a first button is turned, the marker is shifted along the curve. When a second button is turned, the path of the freely movable section is modified, as explained in greater detail below. When a third button is turned, the spatial representation of the curve is modified, for example the spatial representation may be rotated about a predefined axis of rotation. According to a further refinement, the valuator has a plurality of buttons for modifying the representation: one for shifting the representation up or down, one for shifting it to the left or right, one for rotating it about a predefined axis of rotation, and one for zooming in or out of the representation.

Instead of a valuator having at least three rotatable buttons, one having just two buttons and a shift key may be used.

According to an alternative embodiment of the second input device, the second input device includes a keyboard. The marker may be shifted along the curve by striking a specific key, e.g., the arrow keys → or ←. Preferably further keys are also assigned. For example, the path of the freely movable section may be modified by striking another key; and the spatial representation of the curve may be modified by striking another key. Striking yet another key activates a diagnostic tool as described below.

Preferably the device processes numeric input via the keyboard. When the device is in a specific mode, the data processing device positions the position marker as a function of numeric input of this kind.

According to a further embodiment, the second input device is a spacemouse or spaceball. When the spacemouse's button or the spaceball's ball is turned, the marker is shifted along the curve. Preferably, if the button or ball is moved in another direction or a switch is carried out via a key on the input device followed by a subsequent turn of the button, the path of the freely movable section is modified, or the spatial representation of the curve is modified, or a diagnostic tool is activated. A description of a spacemouse and a spaceball may be found at for example http://www.3dconnexion.com/products.htm (status as of Mar. 29, 2004).

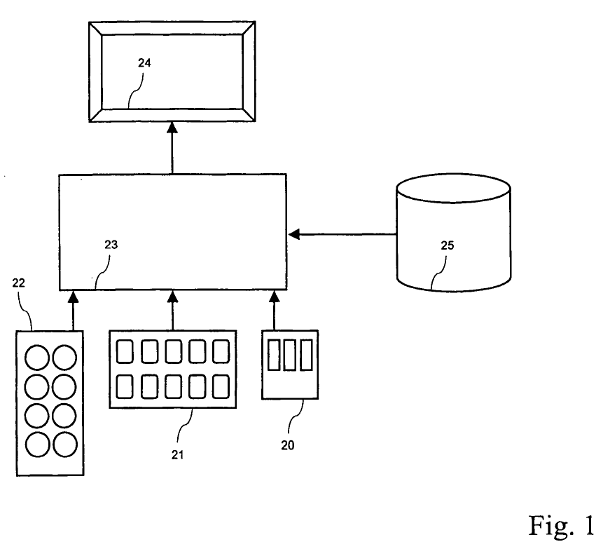

According to a further embodiment, three input devices are used: a mouse, a valuator, and a keyboard. FIG. 1 shows an example of system architecture of the design device. In this example the device includes
a first input device in the form of a data processing mouse 20 having three buttons,
a second input device in the form of a keyboard 21 having keys,
a third input device in the form of a valuator 22 having eight rotatable buttons as shown in FIG. 1,
a data processing device in the form of a PC 23,
an output device in the form of a screen 24,
and a data memory 25, to which PC 23 has read access and in which a surface model of a motor vehicle body is stored; the model is described in greater detail below.

In FIG. 1 the data connections between these components are shown as arrows.

Preferably the design device is designed so that when information is entered using one of the input devices the path of the freely movable section is modified. For example the shape or curvature of the curve is modified; before and also after the modification, the freely movable section and the fixed section together form a smooth curve. Second input device 21 includes for example a plurality of buttons as described above. When the second button is turned, the path of the freely movable section is modified. Or second input device 21 includes a keyboard as described above. When specific keys are struck (e.g., arrow keys for “Page Up” or “Page Down”), the path is modified. Or the path is modified by turning a button of a spacemouse or spaceball.

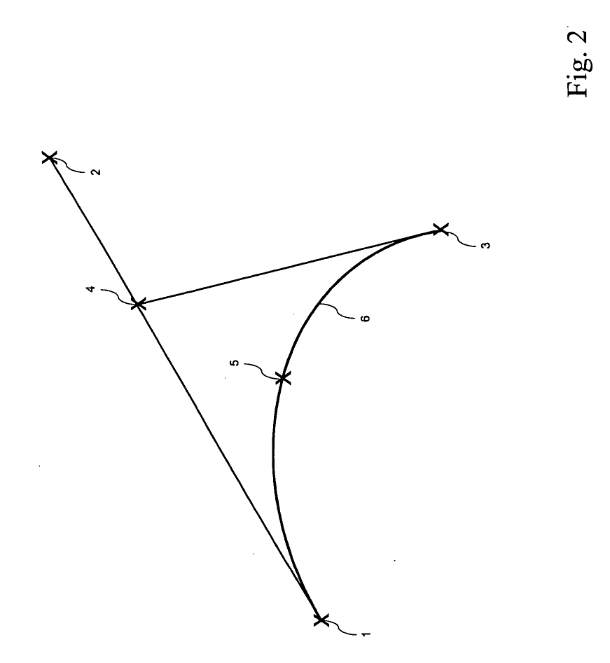

FIG. 2 shows the representation of a curve to be generated and the start of generation of this new curve. Generation of a curve begins when the user selects a point 1, which is displayed on output device 24, e.g., he places the position marker on point 1 using first input device 20 and strikes a key. The device uses this point 1 as the fixed starting point of the curve to be generated. The user then selects a second point 2. The device generates a freely movable section as polyline 15 between first point 1 and second point 2. The user then selects a third point 3. The device generates a curve 6 which extends through point 1 and third point 3. In addition, it selects a fourth point 4 on polyline 15 between first point 1 and second point 2. Curve 6 is generated so that at first point 1 curve 6 is tangential to polyline 15 between first point 1 and second point 2 and furthermore at third point 3 is tangential to the line from point 3 to point 4.

According to an alternative embodiment, the start of a new curve is tangential to an existing object. This existing object may be for example a further curve already generated or a flat or curved surface.

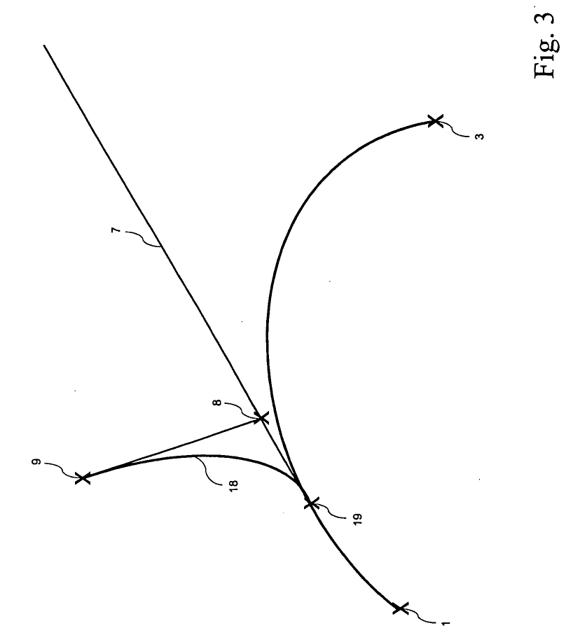

FIG. 3 shows the representation of a further curve to be generated. In the example shown in FIG. 3, curve 6 as shown in FIG. 2 from point 1 to point 3 functions as a further curve. The user selects a further curve that has already been generated, and selects a point 19 on the further curve, which in this case is curve 6, as the starting point of the new curve. The device calculates a tangent 7 to the further curve at point 19, which functions as the starting point of the curve. The user selects a further point 9. The device selects a point 8 on tangent 7 and generates the curve from point 19 to point 9. This curve 18 is tangential to tangent 7 and to the line from point 8 to point 9.

Depending on the information entered via one of the input devices, the device shifts a marker on the representation of curve 6 from first point 1 to third point 3. The user triggers a fix command, e.g., by striking a key. In the example in FIG. 2, when the fix command is received the marker is at point 5. The section of curve 6 between first point 1 and the marker at point 5 is converted into a fixed section. The other section, i.e., the section between point 5 and point 3, is the new freely movable section of the curve.

After this initialization phase has been completed, the curve includes a fixed and a freely movable section. The freely movable section extends from an endpoint of the fixed section to the position marker on the screen. When the position marker is moved, the freely movable section is modified, but the fixed section is not. The path of the fixed section is not changed by extending or modifying the freely movable section.

The freely movable section functions as a preview. Before a partial section is converted into a fixed section, it is analyzable using graphic diagnostic tools, e.g., based on a curvature vector field or control polygon. Analysis tools of this kind are activated via one of the input devices. If the diagnostic tool indicates a bad result, the user may correct the path of the freely movable section.

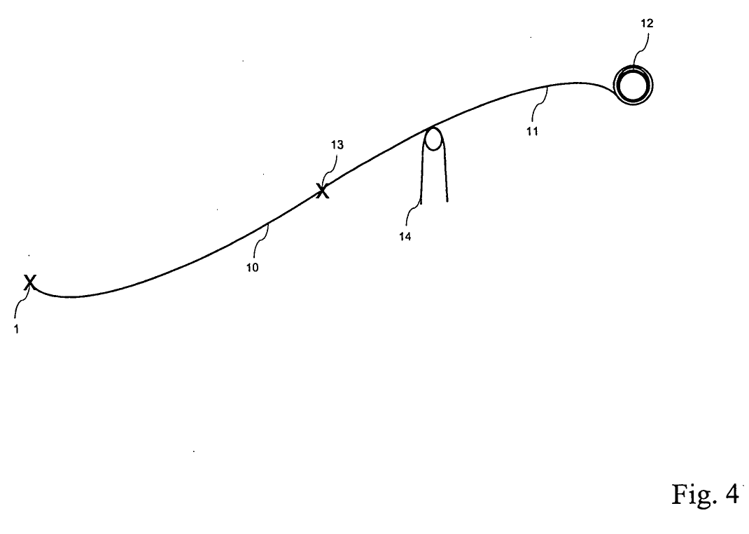

FIG. 4 shows by way of example the representation of a curve having a fixed section 10 and a freely movable section 11. Point 1 functions as the fixed starting point. Fixed section 10 extends from point 1 to point 13. Freely movable section 11 extends from point 13 to position marker 12. Point 13 functions as a connection point between fixed section 10 and freely movable section 11. Depending on the information entered via second input device 21, the device shifts a marker 14 on the representation of the curve. In this example, marker 14 is shown as a finger. Herein, marker 14 may be located in fixed section 10 or in freely movable section 11. If, as shown in FIG. 4, marker 14 is located in freely movable section 11 when the fix command is received, the device converts the partial section of freely movable section 11 between marker 14 and endpoint 13 of existing fixed section 10 into an additional fixed section and joins existing fixed section 10 and the additional fixed section together to form a single new fixed section. The path of existing fixed section 10 remains unchanged when this conversion is performed.

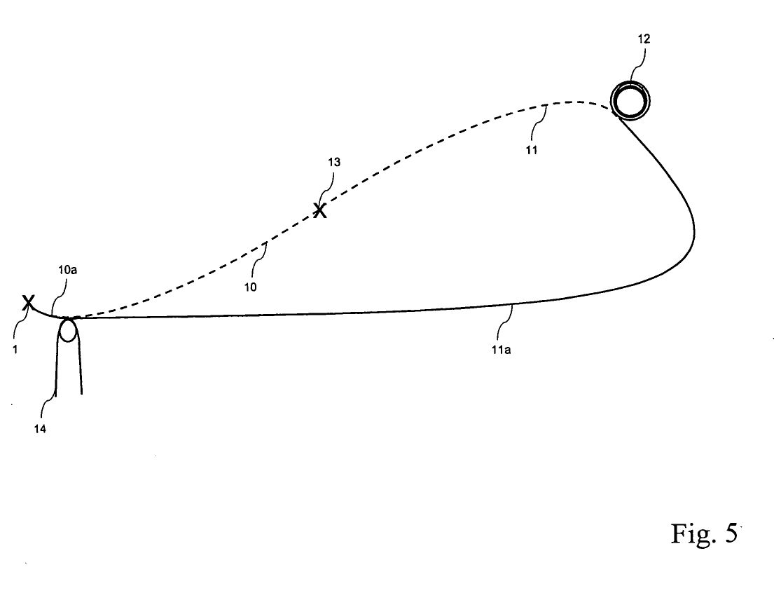

In the example shown in FIG. 5, marker 14 is located in fixed section 10 when the fix command is received. The device deletes the partial section of the fixed section between marker 14 and endpoint 13, which was also the starting point of existing freely movable section 11, and the entirety of freely movable section 11. It generates a new freely movable section 11a, which connects marker 14 to position marker 12 and which along with remaining fixed section 10a forms a smooth curve. In FIG. 5, “old” fixed section 10 and “old” freely movable section 11 are shown as a broken line, and “new” fixed section 10a and “new” freely movable section 11a as a solid line.

In this embodiment, after fixing is released, the path of the curve changes. This matches the physical reality of adhesive tape when it is removed from the surface of an object.

Preferably data processing device 23 displays position marker 12 and marker 14 on output device 24 using two intuitive pictograms. For example in FIG. 4 position marker 12 is shown as a roll of adhesive tape. A roll is generated using for example a plurality of circles, which are preferably arranged concentrically and have different diameters. The roll is shown so that in the representation of the curve on output device 24 freely movable section 11 emerges tangentially from the roll. Marker 14 is shown using for example an image of a pointing finger to represent the fixing function.

The design device is designed so that automatically generated freely movable section 11, which connects fixed section 10 to position marker 12, adjoins fixed section 10 in such a way that the curve is smooth. Moreover, after fixing is released the curve is still smooth. The term “smooth” is defined in Dubbel—Taschenbuch für den Maschinenbau [Dubbel Engineering Handbook], 17^{th} Edition, Springer Verlag 1990, A71, as follows: A curve is considered smooth if it may be expressed with parameters as t→r (t) with t ε [a, b] and r (t)=[x (t), y (t)] (in the case of a curve on a plane) and r (t)=[x (t), y (t), z (t)] (in the case of a curve in space), where x and y (and z) are continuously differentiable to [a, b] and r′ (t)≠0 for all t ε [a, b]. A smooth curve has a tangent at every point and has tangential continuity, i.e., the gradient of the tangent changes continuously in t. Thus at every conversion the section to be converted, which is part of the existing freely movable section, may be described by a function t→r (t) with t ε [a_i, b_i] and r (t)=[x (t), y (t)] (i=1, 2, 3, . . . ). Preferably the intervals follow each other without interruption so that it is true that a_{—}1<b_{—}1=a_{—}2<b_{—}2=a_{—}3<b_{—}3 . . . .

Preferably the design device is designed so that the curve is (n−1) times continuously differentiable at all points, including at the transition between the fixed and the freely movable section. If n=2, the curve has tangential continuity and is therefore smooth. If n=3, the curve has curvature continuity.

According to one embodiment, freely movable section 11 is a polynomial of degree n, i.e., the following is true:
r(t)=c₀+c₁*t+c₂*t²+ . . . +cₙ*tⁿ

Thus when conversion takes place, the partial section to be converted is also a polynomial. The fixed section is therefore a spline of degree n. Splines are described in for example Dubbel (loc cit.), A36. The fixed section in the form of a spline arose via conversion from the freely movable sections which were in the form of polynomials. The positions of marker 14 at the respective instants of conversion constitute the data points of the spline. The curve is (n−1) times continuously differentiable at all points, including at the transition between the fixed and the freely movable section.

Freely movable section 11 is a polynomial of degree n. Thus freely movable section 11 is defined by n+1 vectorial parameters. In two-dimensional space these are 2*(n+1) parameters, in three-dimensional space 3*(n+1) parameters. These n+1 vectorial parameters are defined so that n+1 limiting conditions are met. These n+1 limiting conditions result from the fact that at the transition between fixed section 10 and freely movable section 11 the curve is continuous and (n−1) times continuously differentiable and extends through position marker 12. In the desired representation r (t)=[x (t), y (t)] and r (t)=[x (t), y (t), z (t)] with t E [a, b] for the freely movable section, a is defined by the functional representation of the last fixed section. This last fixed section 10 is represented as t→r (t) with t ε [a_(i−1), b_(i−1)] and r (t)=[x (t), y (t)] or r (t)=[x (t), y (t), z (t)]. For the desired representation t→r (t) with t ε [a_i, b_i] of the freely movable section, it is true that a=a_i=b_(i−1). Furthermore, r (a), r′(a), . . . , r⁽ⁿ⁻¹⁾ (t) are defined by the end of fixed section 10 and by the requirement that the transition be (n−1) times continuously differentiable. r (b) is equal to the position of position marker 12.

One remaining degree of freedom remains, namely the value for b=b_i. This is the value at which position marker 12 is reached. This parameter value b is either set to a fixed value or is determined automatically by data processing device 23 as a function of the current distance between position marker 12 and fixed section 10.

According to one embodiment, the distance between the end of fixed section 10 and position marker 12 is calculated on an ongoing basis, and current distance dist_curr is set as a proportion of distance dist_old at the instant when the last fix command was carried out. Let b_old be the value for b in the representation t→r (t) with t ε [a_old, b_old] of last fixed section 10, and let b_curr be the value to be calculated for the representation of freely movable section 11. In that case, b_curr is calculated on an ongoing basis so that it is true that

$$\frac{b\_curr - a\_curr}{b\_old - a\_old} = \frac{dist\_curr}{dist\_old}$$

The path of freely movable section 11, which is calculated using this calculation method, approximates the path of real adhesive tape without complicated calculations being necessary.

The user may modify this automatically calculated value and thus modify the path of freely movable section 11. Using valuator 22 or by entering numerical information via the keyboard of second input device 21, the user inputs for example a scaling factor between 0.1 and 10 by which the automatically calculated value for (b_curr−a_curr) is multiplied. This embodiment requires much less effort on the part of the user than if he had to manually predefine a new value for b_curr each time. This embodiment is in particular advantageous in the case of a curve having curvature continuity (n=3), because herein the initial curvature of freely movable section 11 is predefined and may be modified by additionally entering the path of the curvature in freely movable section 11.

After receiving an appropriate command, the device calculates a control polygon for the curve or for part of the curve and displays this control polygon on output device 24. For example the device shows the control polygon for the part of the curve between fixed section 10 and marker 14. Preferably this control polygon is determined as follows: The curve and thus the part to be displayed is represented by a function

$$t -> {\sum }_{i = 0}^{n} {{P\_i}_{-}}^{*} {B\_j}^{( n )} ( t ) with t ε [ a , b ]$$

B_{—}0⁽ⁿ⁾, B_{—}1⁽ⁿ⁾, . . . , B_n⁽ⁿ⁾ are n+1 basis functions, e.g., Bernstein polynomials or basis splines of degree n (see Dubbel, loc cit., A37). In the case of cubic basis functions, n=3. P_{—}0, . . . , P_n) are n+1 control points of the curve. The control polynomial is then calculated as a polyline through the n+1 control points and displayed.

An alternative to representing the freely movable section as a polynomial of degree n is to represent it as a spline through a plurality of data points between endpoint 13 of fixed section 10 and position marker 12. The partial curve through these data points is in each respective instance a polynomial of degree n, e.g., n=3. The transition between the two polynomials of a spline at a data point is (n−1) times continuously differentiable. The data points are positioned so that the shape modification energy of the curve, which extends through the endpoint, the data points and position marker 12, is kept to a minimum. Generation of a spline of this kind is described in Dubbel, 17^{th} Edition, A36, left-hand column. In that text, the curve is described by a function y=y (x), and the shape modification energy is calculated via the method 0.5 ∫(M²(x)/(E*I)*y″(x)dx

Alternatively, the shape modification energy may also be calculated as the integral of arc length s. The data points are positioned for example so that the variation of the curvature or curvature variation is minimized. This may be expressed in formulae as follows: If k=k (s) is the curvature, a model for the curvature is minimized, e.g., ∫k²(s) ds or ∫|k(s)|ds or generally ∫∥k(s)∥ds or ∫∥d/dsk(s)∥ds

Preferably the spline that results from repeated conversion is represented as a Bezier spline or B-spline. Both types of representation are commonly used in a wide range of CAD systems. In the case of this embodiment, the curve may be taken over into a design model or surface model without approximation or conversion.

Preferably the curve has a constant width over the entire length. The device modifies a predefined value for the width of the curve as a function of information entered via second input device 21, e.g., as a function of numerical input via the keyboard. Moreover, the color in which the curve is displayed also preferably remains uniform over the entire length. The device modifies this color as a function of the information entered via second input device 21.

The device is preferably designed so that it generates a spatial representation of the curve and displays it on output device 24. The device displays the curve relative to a Cartesian x-y-z coordinate system. The device modifies this spatial representation as a function of information entered by the user. For example, it generates a top view (in the x-y plane), a front view (in the x-z plane), a side view (in the y-z plane), a bottom view (from below in the x-y plane), or a perspective oblique view in a direction of view defined by the user. The view displayed by the device depends on information entered by the user.

Preferably in the initialization phase the device generates the first freely movable section from fixed starting point 1 to position marker 12 so that the section lies completely in the plane of view in question. It subsequently also generates the freely movable section so that it lies completely in the plane of view in question. During the entire curve generation process, information entered via first input device 20 moves position marker 12 in the plane of representation in question and not perpendicular to it. If the user has selected for example the top view, position marker 12 is only moved in the x-y plane. Information entered via second input device 21 shifts position marker 12 perpendicular to the plane of representation, which is not visible in the plane of representation. Freely movable section 11 extends from endpoint 13 of fixed section 10, which as a general rule does not lie in the plane of representation, to position marker 12.

According to a further refinement of this embodiment, the device generates two windows on output device 24 and displays the curve in each of these windows in a view selectable by the user. For example the curve is displayed in one window in top view, i.e., in the x-y plane, and is displayed in the other window in side view, i.e., in the x-z plane. The device generates the freely movable section as just described in the x-y plane, and the user shifts position marker 12 in the window having the top view. In the window having the side view, the device generates a representation which shows the freely movable section as a straight line. For example, if information is entered using a valuator, the user modifies the path of the freely movable section in the side view, i.e., in the x-z plane. As a result of this input, the position of position marker 12 is modified in terms of depth. Herein, “depth” means the third dimension perpendicular to output device 24.

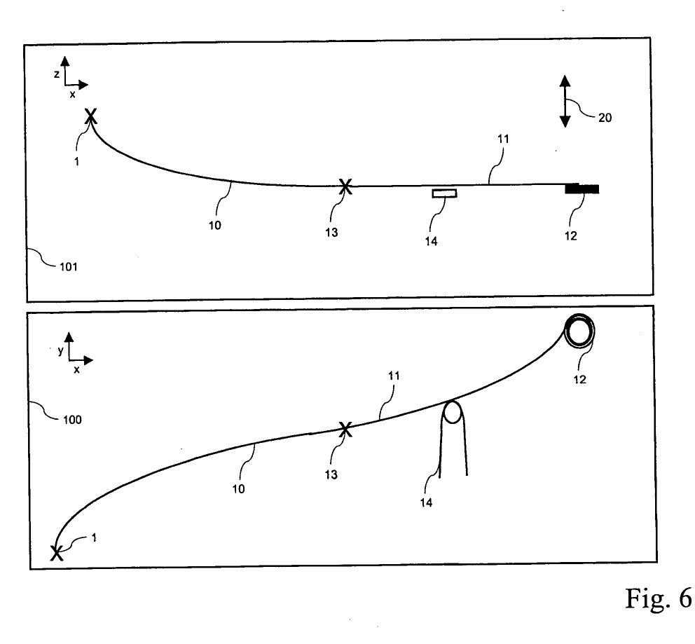

FIG. 6 shows how the device displays the curve in two windows 100 and 101 on output device 24. In both windows, the device displays position marker 12, marker 14, fixed starting point 1, fixed section 10 between points 1 and 13, position marker 12 and freely movable section 11 between point 13 and position marker 12. In this example, the top view (x-y plane) is shown in window 100 and the side view (x-z plane) is shown in window 101. Furthermore, two axes of a Cartesian coordinate system are shown in each respective window to indicate which view is being displayed in which window. One of the windows is selected as the working window; in this example it is window 100. Information entered via second input device 21 moves the freely movable curve in terms of depth, i.e., perpendicular to the plane of window 100. In window 101 this direction is indicated by double-headed arrow 20.

According to a further refinement of this embodiment, the device generates a spatial representation of the curve on a surface model that may be processed by a computer. This model describes at least approximately the surface of a three-dimensional object, e.g., using triangles or other planar elements. The surface model is stored in a data memory 25, to which data processing device 23 has read access. When the device receives a fix command and marker 14 is located in freely movable section 11, the device projects the partial section of freely movable section 11 between marker 14 and existing fixed section 10 in a predefined direction of projection onto the surface model. It is possible that the partial section or a part of the partial section may be projected in the direction of projection onto a plurality of different areas of the surface model, e.g., if the surface model is convex. In this case, the device projects onto the nearest area of the surface model.

The device converts the projected partial section into an additional fixed section and joins the existing section and the additional fixed section together to form a single extended fixed section.

Preferably the device carries out the projection as follows: A given quantity of points on freely movable section 11 between marker 14 and endpoint 13 of the existing fixed section are automatically selected. Preferably the quantity is selected so that the maximum distance between the curve and the polyline through this quantity of points is not greater than a predefined first upper limit. The greater the curvature of the partial section to be projected, the closer the selected points are to one another. These points are projected onto the surface model.

A curve, e.g., a polygon line or a spline, is generated using the projected points. The curve is plotted as an approximation curve through the projected points. For example, it is plotted through all the projected points. Or a spline is generated which is (n−1) times continuously differentiable and is at a minimum distance from the projected points, the distance being measured using a suitable model.

Preferably projection is carried out in the direction of view. The selected and the projected points then differ only with regard to their depth, which is not perceivable in the current view. The shape of the projected partial section does not change

1. An electronic design device comprising:
a first input device;
a second input device;
an output device for displaying a graphic representation including a position marker, wherein information entered using the first input device moves the position marker on the output device; and
a data processing device generating a curve as a function of information entered using the first and second input devices for display on the output device, the curve being generated so as to have a fixed section having a fixed path and a freely movable section connecting an endpoint of the fixed section to the position marker so as to form a smooth transition between the fixed section and the freely moveable section,
wherein a position of a curve marker is shiftable along the curve based on information entered using the second input device, and
wherein, in response to receiving a fix command at a time when the curve marker is disposed along the freely movable section, the data processing device fixes a partial section of the freely movable section between the curve marker and the fixed section and joins the partial section together with the fixed section so as to form an extended fixed section.

2. The device as recited in claim 1, wherein at least one of the first input device and the second input device includes a key, and wherein a striking of the key triggers the fix command.

3. The device as recited in claim 1, wherein the second input device includes a rotatable button, and wherein a rotating of the rotatable button shifts the position of the curve marker along the curve.

4. The device as recited in claim 1, wherein the second input device includes a keyboard including a plurality of keys, and wherein the striking of specific keys of the plurality of keys shifts the position of the curve marker along the curve.

5. The device as recited in claim 1, information entered using one of the input devices modifies a path of the freely movable section.

6. The device as recited in claim 1, wherein, in response to receiving a fix command at a time when the curve marker is disposed along the fixed section, the data processing device deletes a partial section of the fixed section between the curve marker and the freely moveable section as well as the freely moveable section and generates a new freely movable section between the curve marker and the position marker so as to form a smooth transition between the new freely movable section and remaining portion of the fixed section.

7. The device as recited in claim 1, wherein the freely movable section is a polynomial and the fixed section is a spline.

8. The device as recited in claim 1, wherein the data processing device generates a spatial representation of the curve in a Cartesian coordinate system.

9. The device as recited in claim 8, wherein the data processing device generates two spatial representations of the curve from two different directions of view and displays them on the output device.

10. The device as recited in claim 9, wherein the data processing device displays the two representations in two different windows of the output device.

11. The device as recited in claims 1, wherein the data processing device has read access to a data memory holding a surface model of a three-dimensional object that is processable by a computer, and wherein in response to receiving the fix command at a time when the curve marker is disposed along the freely movable section the data processing device projects the partial section of the freely movable section between the curve marker and the fixed section in a predefined projection direction onto the surface model, converts the projected partial section into an additional fixed section, and joins the fixed section and the additional fixed section together to form a single extended fixed section.

12. A method for generating and displaying a curve that is processable by a computer, the method comprising:
moving a position marker on an output device as a function of information entered via a first input device;
generating and displaying the curve having a fixed section including a fixed path and a freely movable section on the output device as a function of information entered via the first input device and a second input device, wherein the freely movable section is generated so as to connect an endpoint of the fixed section to the position marker and so as to form a smooth transition between the fixed section and the freely moveable section;
shifting a position of a curve marker along the curve as a function of information entered via the second input device;
in response to receiving a fix command at a time when the curve marker it disposed along the freely moveable section, fixing a partial section of the freely moveable section between the curve marker and the fixed section and joining the partial section together with the fixed section so as to form an extended fixed section.

13. The method as recited in claim 12, further comprising, in response to receiving a fix command at a time when the curve marker is disposed along the fixed section, deleting a partial section of the fixed section between the curve marker and the freely moveable section as well as the freely moveable section and generating a new freely movable section between the curve marker and the position marker so as to form a smooth transition between the new freely movable section and remaining portion of the fixed section.

14. The method as recited in claim 12, wherein a spatial representation of the curve is generated in a Cartesian coordinate system.

15. The method as recited in claim 14, wherein two spatial representations of the curve from two different directions of view are generated and displayed on the output device.

16. The method as recited in claim 12, wherein the fixing of the partial section includes projecting the partial section of the freely moveable section onto a surface model of a three-dimensional object that is processable by a computer, converting the projected partial section into an additional fixed section, and joining the fixed section and the additional fixed section together to form a single extended fixed section.

17. A computer program product which can be loaded directly into the internal memory of a computer and includes software sections via which a method as recited in claim 12 can be carried out if the product runs on a computer.

18. A computer program product that is stored on a medium which is readable by a computer and which has program means that are readable by a computer which cause the computer to carry out a method as recited in claim 12.
